(19) United States
(12) Patent Application Publication
(10) Pub. No.: US 2008/0319104 A1
(43) Pub. Date: Dec. 25, 2008

(54) Hardening Dental Materials Featuring Adjustable Translucence

(76) Inventors: Simone Klapdohr, Rankweil (AT); Norbert Moszner, Triesen (LI); Karin Vogel, Gamprin-Bendern (LI); Volker Rheinberger, Vaduz (LI); Peter Burtscher, Rankweil (AT)

Correspondence Address: FRIEDRICH KUEFFNER, 317 MADISON AVENUE, SUITE 910, NEW YORK, NY, 10017, US

(21) Appl. No.: 11/547,939

(22) PCT Filed: Apr. 6, 2005
(86) PCT No.: PCT/EP05/51594

§ 371 Date: Jul. 30, 2007

(30) Foreign Application Priority Data
Apr. 7, 2004 (DE) 10 2004 017 124.6

(51) Int. Cl. A61K 6/08

(52) U.S. Cl. 523/117; 523/116

(57) ABSTRACT

The present invention relates to a curable dental material having adjustable translucence and high opalescence containing
I. at least one monomer or one monomer mixture having a refractive index of <1.45,
II. at least one opalescent filler having a refractive index of <1.45,
III. further customary fillers or filler mixtures and
IV. polymerization initiators, stabilizers, dyes or mixtures of one or more of these substances.

The invention relates to curable dental materials having an adjustable translucence and high opalescence, in particular highly esthetic filling materials.

From the prior art, many dental materials are known to which properties are ascribed which should qualify these as esthetic materials. In the literature, scarcely any information is found about such properties, in particular about opalescence or translucence and their selective adjustment and variations.

The aim of dental restorations is to allow the restoration to look like a natural tooth. The natural tooth shows various translucences, fluorescence and opalescence. As a result, the tooth appears alive. There is permanently the endeavor to imitate not only the tooth color and the color gradient in the restorations, to improve them using more colors and all sorts of restoration techniques, but also to integrate fluorescent effects into the restoration material. This was attempted in various ways in the 1990s by, for example, adding blue dyes or alternatively microfine titanium dioxide (e.g. EP 533 434) to the conventional material.

The natural color of the tooth is determined by the dentine. The opalescence effect in the tooth results due to the almost transparent enamel layer. Owing to its crystalline structure, it refracts the light in such a way that opalescence occurs.

From U.S. Pat. No. 6,323,367 (Kobashigawa et al.), opalescent fillers for dental materials are known which are composed of a mixture of a ground (customary and known for a long time) translucent glass filler and a colloidal filler. Here, the refractive indices of the fillers and of the polymerizable monomers used are coordinated with one another such that a translucent material can be prepared. The refractive index of the monomer mixture employed is between 1.45 and 1.60. The difference in the refractive indices between monomer mixture and filler is +/−0.04.

The aim of the invention is the preparation of an opalescent dental material in which the achievable transparencies and opalescences in the material are selectively adjustable.

The object according to the invention is achieved by a curable dental material comprising
I. at least one monomer or one monomer mixture having a refractive index of <1.45,
II. at least one opalescent filler having a refractive index of <1.45,
III. further customary fillers or filler mixtures
IV. polymerization initiators, stabilizers, dyes or mixtures of one or more of these substances and
V. if appropriate further customary additives.

Preferably, the curable dental materials according to the invention contain
I 10 to 85% by weight of polymerizable monomer or polymerizable monomer mixture,
II 10 to 70% by weight of opalescent filler,
III 5 to 60% by weight of further customary fillers or filler mixtures and
IV 0.01 to 5% by weight of polymerization initiators, stabilizers, dyes or mixtures of one or more of these substances.

The dental materials are particularly suitable as cements, veneer materials and in particular filling composites.

A particularly preferred cement contains:
I 10 to 85% by weight, in particular 20 to 50% by weight, of polymerizable monomer or polymerizable monomer mixture,
II 10 to 70% by weight, in particular 10 to 40% by weight, of opalescent filler,
III 5 to 50% by weight, in particular 10 to 40% by weight, of further customary fillers or filler mixtures and
IV 0.01 to 5% by weight, in particular 0.1 to 2.0% by weight, of polymerization initiators, stabilizers, dyes or mixtures of one or more of these substances,
   in each case based on the total mass of the cement.

A particularly preferred veneer material contains:
I 10 to 50% by weight, in particular 10 to 30% by weight, of polymerizable monomer or polymerizable monomer mixture,
II 30 to 70% by weight, in particular 40 to 70% by weight, of opalescent filler,
III 20 to 50% by weight, in particular 30 to 40% by weight, of further customary fillers or filler mixtures and
IV 0.01 to 5% by weight, in particular 0.1 to 2.0% by weight, of polymerization initiators, stabilizers, dyes or mixtures of one or more of these components,
   in each case based on the total mass of the veneer material.

A particularly preferred filling material contains:
I 10 to 85% by weight, in particular 15 to 60% by weight, of polymerizable monomer or polymerizable monomer mixture,
II 15 to 65% by weight, in particular 40 to 65% by weight, of opalescent filler,
III 5 to 60% by weight, in particular 10 to 50% by weight, of further customary fillers or filler mixtures and
IV 0.01 to 5% by weight, in particular 0.1 to 2.0% by weight, of polymerization initiators, stabilizers, dyes or mixtures of one or more of these components,
   in each case based on the total mass of the filling material.

The aim of the invention is achieved by the suitable choice of the filler components taking into consideration the refractive indices of fillers and monomer mixture. The dental material according to the invention surprisingly exhibited adjustable values for the translucence and opalescence in a manner which is not known from the present state of the art.

According to the invention, mixtures of suitable free radical-polymerizable monofunctional and/or poly-functional monomers, in particular di-, tri- and tetra-functional, very particularly preferably difunctional, crosslinker monomers, are preferably used for component I.

Monofunctional monomers are understood as meaning compounds having a free radical-polymerizable group, polyfunctional monomers as compounds having two or more free radical-polymerizable groups. For the preparation of adhesives, coating materials and dental materials, crosslinking di- or polyfunctional acrylates or methacrylates, such as, for example, bisphenol A di(meth)acrylate, bis-GMA (the addition product of methacrylic acid and bisphenol A diglycidyl ether), UDMA (the addition product of hydroxyethyl methacrylate and 2,2,4-trimethyl hexamethylene-diisocyanate), di-, tri- or tetraethylene glycol di(meth)acrylate, decanediol di(meth)acrylate, tri-methylolpropane tri(meth)acrylate and pentaerythritol tetra(meth)acrylate are especially suitable. The compounds butanediol di(meth)acrylate, 1,10-decanediol di(meth)acrylate and 1,12-dodecanediol di(meth)-acrylate, accessible by esterification of (meth)acrylic acid with the corresponding diols, and also di- and polyfunctional 2-vinylcyclopropane derivatives which are accessible by reaction of 1-methoxycarbonyl-2-vinyl-cyclopropane-1-carboxylic acid with di- or polyhydric OH or NH₂ compounds as coupling component, i.e., for example, ethylene glycol, di- or triethylene glycol, butylene glycol, 1,6-hexanediol, glycerol, pentaerythritol or glucose, and also hydroquinone, resorcinol, pyrocatechol or pyrogallol, ethylenediamine, propylene-diamine, hexamethylenediamine, o-, p- or m-phenylene-diamine are likewise suitable.

Further polyfunctional free radical-polymerizable monomers which are particularly suitable as crosslinker monomers are urethanes of 2-(hydroxymethyl)acrylic acid ethyl ester and diisocyanates, such as, for example, 2,2,4-trimethylhexamethylene diisocyanate or isophorone diisocyanate, crosslinking pyrrolidones, such as, for example, 1,6-bis(3-vinyl-2-pyrrolindonyl)hexane or commercially accessible bisacrylamides, such as methylene- or ethylenebisacrylamide or bis(meth)-acrylamides, such as, for example, N,N′-diethyl-1,3-bis(acrylamido)propane, 1,3-bis(methacrylamido)propane, 1,4-bis(acrylamido)butane or N,N′-bis(acryloyl)-piperazine, which can be synthesized by reaction of the corresponding diamines with (meth)-acryloyl chloride. These compounds are moreover distinguished by a relatively high resistance to hydrolysis.

Preferred monofunctional free radical-polymerizable monomers which are particularly suitable as thinner monomers are hydrolysis-stable mono(meth)acrylates, such as, for example, mesityl methacrylate, or 2-(alkoxymethyl)acrylic acids, such as 2-(ethoxymethyl)-acrylic acid, 2-(hydroxymethyl)acrylic acid, N-mono- or -disubstituted acrylamides, such as, for example, N-ethylacrylamide, N,N-dimethylacrylamide, N-(2-hydroxy-ethyl)acrylamide or N-(2-hydroxyethyl)-N-methylacryl-amide and also N-monosubstituted methacrylamides, such as, for example, N-ethylmethacrylamide or N-(2-hydroxy-ethyl)methacrylamide or alternatively tert-butyl methacrylate, allyl methacrylate, isooctyl acrylate, 2-(2-ethoxyethoxy)ethyl acrylate, octyldecyl acrylate, lauryl methacrylate, tridecyl methacrylate, propylene glycol monomethacrylate, 2-ethoxyethyl acrylate, tert-butyl acrylate, lauryl acrylate and isobornyl acrylate.

For the reduction of the surface energy, it is known to employ fluoro-substituted monomers additionally in the mixtures or on their own, since as a result the tendency for plaque formation and accumulation on the tooth surface can be reduced.

Preferred fluorinated monofunctional monomers are 2,2,2-trifluoroethyl(meth)acrylate, pentafluoromethyl methacrylate, 2-(pentafluorobutyl)ethyl(meth)acrylate, 2,2,3,3-tetrafluoropropyl(meth)acrylate, 3-(penta-fluorobutyl)-2-hydroxypropyl(meth)acrylate, perfluoro-cyclohexylmethyl methacrylate, 3-(perfluorohexyl)-2-hydroxypropyl(meth)acrylate, 2-(perfluoro-3-methyl-butyl)ethyl methacrylate, 3-(perfluoro-3-methylbutyl)-2-hydroxypropyl(meth)acrylate, 1H,1H,5H-octafluoro-pentyl (meth)acrylate, 1H,1H,2H,2H-pentafluorodecyl acrylate, 1H,1H-perfluoro-n-decyl(meth)acrylate, 2-(perfluorodecyl)ethyl(meth)acrylate, 2-(perfluoro-9-methyldecyl)ethyl(meth)acrylate, 2-(perfluoro-5-methylhexyl)ethyl(meth)acrylate, 2-(perfluoro-7-methyloctyl)ethyl(meth)acrylate, 1H,1H,7H-dodeca-fluoroheptyl (meth)acrylate, 1H,1H-perfluorooctyl (meth)acrylate, 1H,1H,2H,2H-perfluorooctyl(meth)-acrylate, 1H,1H,9H-hexadecafluorononyl(meth)acrylate and 1H,1H,1H,11H-eicosafluoroundecyl(meth)acrylate and 1H,1H,2H,2H-pentafluorodecyl acrylate.

Preferred fluorinated crosslinker monomers are fluorinated triethylene glycol dimethacrylate (TEGDMA-F), 2,2,3,3-tetrafluoro-1,4-butanediol dimethacrylate, 1H,1H,6H,6H-perfluoro-1,6-hexanediol di(meth)acrylate, 1H,1H,10H,10H-perfluorodecanediol di(meth)acrylate, 2,2,3,3,4,4,5,5,6,6,7,7-dodecafluoro-1,8-octanediol di-(meth)acrylate and fluorinated bis-GMA {bis-GMA-F: 2,2-bis[(4-(2-hydroxy-3-methacryloyloxy)phenyl]hexafluoro-propane.

The refractive index of the monomer or of the monomer mixtures employed is <1.45 measured at 25° C. Preferably, the refractive index is 1.380 to 1.449. A refractive index of 1.420 to 1.447 is particularly preferred.

For polymerization, these monomers, as the main constituent of dental materials, are mixed for free-radical polymerization with an initiator and preferably also with additional monomers, fillers and optionally further auxiliaries. The compositions thus obtained can be cured by free-radical polymerization. The invention relates both to the curable compositions and the cured products.

The known initiators for hot curing, cold curing and photocuring are suitable as initiators for free-radical polymerization. Suitable initiators are described, for example, in the Encyclopedia of Polymer Science and Engineering, Vol. 13, Wiley-Intersci. Pub., New York etc. 1988, p. 754 ff.

Preferred initiators are azo compounds, such as azo-bis(isobutyronitrile) (AIBN) or azobis(4-cyanovaleric acid) or peroxides, such as dibenzoyl peroxide, di-lauryl peroxide, tert-butyl peroctoate, tert-butyl per-benzoate or di-(tert-butyl)peroxide.

Suitable initiators for hot curing are particularly benzopinacol and 2,2′-di(C₁-C₈-alkyl)benzopinacols.

Suitable photoinitiators for the UV or visible range are described by J. P. Fouassier, J. F. Rabek (ed.), Radiation Curing in Polymer Science, London and New York 1993, p. 155 to p. 237. Preferred photoinitiators are benzoin ethers, dialkylbenzil ketals, dialkoxy-acetophenones, acylphosphine oxides, bisacylphosphine oxides, α-diketones such as 10-phenanthrenequinone, diacetyl, furil, anisil, 4,4′-dichlorobenzil and 4,4′-dialkoxybenzil and camphorquinone.

For the preparation of dental materials, dibenzoyl peroxide, camphorquinone and acylphosphine oxides are preferred.

For acceleration of the initiation by peroxides or α-diketones, combinations with aromatic amines are particularly suitable. Accelerators which are moreover employable are redox systems, in particular combinations of benzoyl peroxide, lauroyl peroxide or camphorquinone with amines, such as N,N-dimethyl-p-toluidine, N,N-dihydroxyethyl-p-toluidine, p-dimethyl-aminobenzoic acid ethyl ester or structurally related amines.

Moreover, suitable redox systems are also those which, in addition to peroxide, contain ascorbic acid, a barbiturate, a sulfinic acid or mercapto compounds, such as mercaptobenzothiazole, 2-mercaptobenzoxazole or 2-mercaptobenzimidazole, as reductants.

For improvement of the mechanical properties or for adjustment of the viscosity, various organic and inorganic particles or fibers can be used as fillers in dental materials.

Compared with the prior art, curable dental materials according to the invention are made available from which cured dental materials having considerably higher opalescence can be produced. By the combination of the fillers of components II and III, opalescence and translucence can be selectively adjusted.

The opalescent fillers employed according to the invention in component II differ from those of the prior art in that not a ground filler, but a spherical filler is employed. The particle size distribution of the fillers employed according to the invention is distinctly narrower than that in U.S. Pat. No. 6,232,367.

The opalescent fillers employed according to the invention are if possible monodisperse, almost ideally spherical particles. These cannot be obtained by means of a grinding process, as also described in the patent U.S. Pat. No. 6,323,367. According to the invention, the particles are therefore prepared by means of the known sol-gel process, namely following the Stöber process [cf. W. Stöber et al. in J. Colloid and Interface Science 26, 62 (1968) and 30, 568 (1969), U.S. Pat. No. 3,634,588, EP 0 216 278]. The mean particle size is 230±50 nm. Mean particle sizes of 230±20 nm are particularly preferred.

The particles of the opalescent filler must not scatter strongly from the mean value. According to the invention, the standard deviation of the particles from the mean value is less than 7%, particularly preferably less than 5%.

Before use in the dental material, the particles are preferably silanized with customary polymerizable silanes. This causes a better interlocking between the matrix and filler after polymerization and increases the mechanical properties of the cured material. The filler can, however, also be incorporated and distributed better in the matrix by silanization. Surprisingly, the opalescence effect was also increased thereby.

The refractive index of the opalescent filler employed according to the invention is <1.45. Particularly preferably, refractive indices of the opalescent filler are between 1.40 and 1.45, very particularly preferably between 1.41 and 1.44.

The differences in the refractive indices of components I and II are ≦0.04, preferably ≦0.02, particularly preferably ≦0.01.

According to the invention, further customary fillers or filler mixtures can be employed in component III. These preferably have a mean particle size of between 5 and 1000 nm, particularly preferably 40 nm and 500 nm, very particularly preferably 80 to 300 nm and highly preferably 80 to 120 nm. The refractive index of the further fillers is preferably above that of the components I and II. Preferably, the refractive index is 1.45 to 1.55, preferably 1.46 to 1.52, very particularly preferably 1.46 to 1.48.

Preferred fillers of component III for the preparation of dental materials such as fixing cements, coating materials or filling materials are amorphous, spherical materials based on oxides such as SiO₂, ZrO₂, TiO₂ or mixed oxides of SiO₂, ZrO₂ and/or TiO₂ having a mean primary particle size of 0.005 to 1.0 μm, in particular of 0.01 to 0.3 μm, nanoparticulate or microfine fillers, such as pyrogenic silica or precipitated silica, and X-ray-opaque fillers, such as ytterbium fluoride, nanoparticulate tantalum (V) oxide or barium sulfate.

The nanofillers which can be employed according to the invention have a mean particle size of ≦40 nm, preferably 5 to 25 nm, particularly preferably 10 to 20 nm.

The content of the nanofiller in the monomer can be 2-10% by weight, preferably 5 to 10% by weight, based on the monomer.

Preferably, the nanofillers employed are spherical, nonagglomerated nanofillers. For example, an “organosol” from Clariant or Hanse Chemie can be employed as a filler. In this organosol, the particles are surface-coated with a polymerizable silane and dispersed in a polymerizable monomer.

Organic fillers which are used are very often also “composite fillers”, which consist of a monomer or monomer mixture and one or more inorganic fillers, such as, for example, ground glasses or various SiO₂ modifications or X-ray-opaque fillers. These mixtures are polymerized in a suitable manner, preferably by heat curing, and subsequently ground to a suitable particle size; particle sizes of about 0.1 to 50 μm, particularly preferably 0.2 to 20 μm, are preferred here.

The content of the organic radicals in the inorganic opalescent filler is preferably ≦5% by weight, particularly preferably ≦3% by weight.

Moreover, the compositions according to the invention can, if required, contain further auxiliaries, in particular stabilizers, UV absorbers, dyes, pigments and/or lubricants. Stabilizers are understood as meaning those which prevent premature polymerization and thus especially increase the storage stability of monomer mixtures and materials without, however, adversely affecting the properties of the cured materials. Preferred stabilizers are hydroquinone monomethyl ether (MEHQ) and 2,6-di-tert-butyl-4-methyl-phenol (BHT).

The compositions according to the invention are particularly suitable as dental materials, in particular as filling materials, fixing cements or coating materials, and materials for inlays/onlays, teeth or veneer materials for crowns and bridges. In addition to a low polymerization shrinkage and excellent mechanical properties, they are distinguished especially by their high translucence and adjustable opalescence.

The invention is explained in more detail below with reference to examples, the invention not being restricted to the examples mentioned.

### EXAMPLE 1

### Preparation of an Organosol “UDMA silanized with 40% by weight of SiO₂ (13 nm)”

### a) Silanization of the Nanoparticles

Starting from 120 g of Highlink® OG 502-31 (Clariant), 5.36 g of gamma-methacryloxypropyltrimethoxysilane (A 174) were added to a colloidal solution of SiO₂ having a mean particle size of 13 nm (30% by weight SiO₂ in isopropanol), and the mixture was stirred at room temperature for 2 minutes. Subsequently, 1.17 g of 0.5 N HCl were added and the mixture was stirred at room temperature for 6 hours. The sol obtained in this way contained 28.4% by weight of SiO₂ particles.

[In the surface modification, the original sol was diluted from 30% by weight to 28.4% by weight by the water and the silane. Of the silanized organosol, only the surface-modified SiO₂ in the lower mixture reacts with the monomer, all other volatile constituents are removed (isopropanol, the alcohol eliminated during the silanization and the water) such that the final composition contains 40% by weight of SiO₂ and just under 60% by weight of urethane dimethacrylate (UDMA). A small amount of silane from the surface modification is still present].

Incorporation of the Particles into UDMA to 40% by Weight

50.13 g of UDMA were added to 126.53 g of the organosol described above and stirred until the mixture was homogeneous. After addition of a stabilizer (e.g. MEHQ), the volatile constituents were removed at 40° C. on a rotary evaporator, and 90 g of a translucent highly viscous oil were obtained, which contains 40% by weight of SiO₂ and 55.7% by weight of UDMA.

The composition of the solution is

| Mass | Content | |
|---|---|---|
| (in g) | (in % by weight) | Component |
| 36.0 | 40.0 | SiO₂ |
| 3.87 | 4.3 | Condensed silane (on |
| | | complete condensation) |
| 50.13 | 55.7 | UDMA |

### EXAMPLE 2

### Preparation of the Opalescent Filler from SiO₂

A hydrolysis mixture of 71.4 g of water, 376.2 g of ethanol and 9.0 g of 25% strength by volume ammonia solution was prepared. This hydrolysis mixture was warmed to 40° C., and 26.4 g of tetraethoxysilane were added with intensive stirring. This mixture was stirred further at 40° C. for 2 hours. In the course of this, a sol resulted having an SiO₂ primary particle size of about 90 to 115 nm.

Over a period of 10 to 12 hours, a hydrolysis mixture of 271.4 g of water, 1433.8 g of ethanol and 46.8 g of 25% strength by volume ammonia solution and in parallel 216.7 g of tetraethoxysilane were added to this sol. The size of the particles resulting here was checked periodically by means of SEM.

A mixture of 0.96 g of gamma-methacryloxypropyl-trimethoxysilane in 59.56 ml of ethanol was added dropwise at 40° C. to the particles thus obtained over a period of about 4 hours. This mixture was stirred at 40° C. for a further 2 hours. In the course of this, the particles became surface-modified with the silane. Subsequently, the sol was freed of the volatile constituents on a rotary evaporator at 40° C. The white powder obtained was finally dried at a pressure of 0.1 mbar and a temperature of 110° C. for 12 hours. The size of the particles was 215 nm with a standard deviation of 4.6%.

### Measurement Principles/Measurement Methods

The transparency is measured by means of a CT-310 chromameter from Minolta. Here, the light transmitted by a defined test article (d=1 mm) in water relative to the light transmitted in a pure water sample is measured. The value obtained is stated in percent. The transparency of the natural enamel varies between 45 and 80%. (See also U.S. Pat. No. 6,323,367).

The particle size of the spherical filler particles was determined by means of scanning electron microscopy. The width of variation was determined by measurement of a statistically significant amount.

The opalescence is measured by means of the coupled CT-310 3-filter transmission system and CR-300 3-filter emission system, both apparatuses from Minolta. Here, the CIELAB assessment system used has been described in detail in U.S. Pat. No. 6,232,367 (column 6 f.).

### Fillers:

All fillers which were used in the following examples were surface-treated, if this did not take place in the preparation process, in a mixer by addition of water and a silane having a polymerizable group (gamma-methacryloxypropyl-trimethoxysilane (A-174)). The silane content varies, depending on the BET surface area of the filler, between 4 and 10% by weight, based on the total mass of the respective filler.

### EXAMPLE 3

The compositions listed in Table 1 were used for the preparation and the comparison of the various dental materials (all data, if not stated otherwise, in % by weight):

TABLE 1

| | Monomer mixture | | |
|---|---|---|---|
| | 1 | 2 | 3 |
| V-466 | 60 | 60 | 60 |
| UDMA | 40 | 20 | — |
| 40% by weight SiO₂ | — | 20 | 40 |
| in UDMA | | | |
| Refractive index [%] | 1.4466 | 1.4439 | 1.4406 |

Between 0.01 and 5.0% by weight of initiators, stabilizers, accelerators or dyes, which are adequately known from the prior art, are added to these monomer mixtures. These additions are dissolved in the monomer mixture by stirring at room temperature.

The monomer mixture is introduced into a planetary mixer and the fillers are added in portions over a period of 1 hour. The resulting mixture is subsequently kneaded for a further hour. Afterward, the mixture obtained is deaerated during 15 minutes and to a vacuum of about 160 mbar.

For the measurement of transparency and opalescence, a mixture of a monomer mixture 2 from Table 1 and a spherical filler having a differing primary particle size (always in the ratio 35% by weight of monomer mixture and 65% by weight of filler) is prepared. A test mold (Ø=20 mm, H=1 mm) is slightly overfilled with the mixtures and compressed using 20 bar. Subsequently, the test article is exposed in a Spektramat® light oven from Ivoclar Vivadent AG for 2 times 3 min, demolded and the transparency and opalescence are measured.

TABLE 2

| | Particle size | Transparency | Opalescence |
|---|---|---|---|
| Sample | [in nm] | [in %] | [in %] |
| No. 1 | 40* | 46.3 | 2.96 |
| No. 2 | 230** | 41.0 | 39.0 |
| No. 3 | 500*** | 6.48 | 6.25 |

*OX 50 Degussa AG
**filler according to the invention
***Monosphere ® 500 Degussa AG

It can be concluded from the results listed above that the opalescence only appears in a narrow size range of the filler particles. On the basis of the correspondence of refractive index of monomer mixture 1 and the 40 nm particles, although the transparency is very high, the opalescence is only very low. Only with the 230 nm particles according to the invention is a high transparency and at the same time a high opalescence achievable.

### EXAMPLE 4

### Preparation of a Highly Transparent and Opalescent Composite

The samples were prepared according to the above description. The filler used is the filler according to the invention having a primary particle size of about 230 nm from sample 2. The composition of these samples always consists to 35% by weight of the monomer mixture and to 65% by weight of the filler.

TABLE 3

| | | Refractive | | |
|---|---|---|---|---|
| | Monomer | index | Transparency | Opalescence |
| Sample | mixture | monomer | [in %] | [in %] |
| No. 4 | 1 | 1.4466 | 26.5 | 32.2 |
| No. 5 | 2 | 1.4439 | 49.2 | 40.2 |
| No. 6 | 3 | 1.4406 | turbid | turbid |

By means of a defined content of monomer containing nanoscale particles in the monomer, the transparency of the resulting mixture can be increased. If, however, the limit is still exceeded above, as a result of incompatibilities of the mixture clouding occurs. The refractive index of the monomer mixture must be below 1.45 in order to achieve a high transparency and at the same time a high opalescence.

The enamel layer of the natural tooth possesses opalescent properties. Additionally, it is highly transparent. Depending on the application of the opalescent composite as an esthetic filling material or as a flowable material, the requirement for opalescence is different. Since the uppermost layer of the restoration is concerned, sometimes the transparency must be kept high and the opalescence is reduced stepwise.

### EXAMPLE 5

### Composite Having High Transparency and Graduated Opalescence

For the measurements, test articles of monomer mixture 2 (35% by weight) and two spherical fillers (together 65% by weight) were prepared:

TABLE 4

| | Opalescent | Filler | | |
|---|---|---|---|---|
| | filler | NX 10 | | |
| | (230 nm) | sil.**** | | |
| | [in % by | [in % by | Transparency | Opalescence |
| Sample | weight] | weight] | [in %] | [in %] |
| No. 7 | 65 | 0 | 63.9 | 39.7 |
| No. 8 | 55 | 10 | 57.0 | 37.9 |
| No. 9 | 45 | 20 | 52.4 | 34.8 |
| No. 10 | 10 | 55 | 51.6 | 22.5 |

****NX 10 sil. silanized spherical particles of silica having a primary particle size of about 100 nm from Degussa AG

By the addition of amounts of a further spherical filler having a particle size of about 100 nm, the opalescence can be reduced as required without changing the transparency significantly. The differences between a transparency of 50% and of 70% are practically imperceptible to the human eye.

### EXAMPLE 6

### Preparation of a Flowable Composite

For the preparation of the flowable composite, the monomer mixture 2 of Table 1 and the opalescent filler having a primary particle size of about 230 nm were used.

TABLE 5

| | Filler content (230 nm) | Opalescence |
|---|---|---|
| Sample | [in % by weight] | [in %] |
| No. 11 | 20 | 5.5 |
| No. 12 | 40 | 12.0 |
| No. 13 | 50 | 19.5 |
| No. 14 | 60 | 25.2 |
| No. 15 | 65 | 38.6 |

In addition to the possibility of the selective adjustment of the opalescence acc. to Example 4, this can also be influenced by means of the content of the monomer mixture. A high monomer content here reduces the opalescence.

1. A curable dental material having adjustable translucence and high opalescence comprising
I. at least one fluoro-substituted monomer monomer or one monomer mixture comprising a fluoro-substituted monomer having a refractive index of <1.45,
II. at least one opalescent filler having a refractive index of <1.45,
III. further customary fillers or filler mixtures and
IV. polymerization initiators, stabilizers, dyes or mixtures of one or more of these substances.

2. The dental material as claimed in claim 1, wherein the monomer or the monomer mixture has a refractive index of 1.380 to 1.449 and the opalescent filler has a refractive index of 1.40 to 1.45.

3. The dental material as claimed in claim 1, wherein the difference between the refractive indices of the components I. and II. is ≦0.04, preferably ≦0.02.

4. The dental material as claimed in claim 1, wherein it contains
I. 10-85% by weight of polymerizable monomer or polymerizable monomer mixture,
II. 10-70% by weight of an opalescent filler,
III. 5-60% by weight of further customary fillers or filler mixtures,
IV. 0.01-5% by weight of polymerization initiators, stabilizers, dyes or a mixture of one or more of the substances mentioned.

5. The dental material as claimed in claim 1, wherein it contains spherical fillers or filler mixtures in component II.

6. The dental material as claimed in claim 1, wherein the fillers in component II have a mean particle size of 230 nm +/−50 nm, preferably of 230+/−20 nm.

7. The dental material as claimed in claim 1, wherein the content of the organic radicals in the inorganic opalescent filler is ≦5% by weight.

8. The dental material as claimed in claim 1, wherein the fillers or filler mixtures in accordance with component III have a mean particle size of 5 nm-1000 nm, preferably of 40 to 500 nm.

9. The dental material as claimed in claim 1, wherein the fillers contained are amorphous spherical materials based on oxides, preferably SiO₂, ZrO₂, TiO₂ or mixtures of these substances.

10. The dental material as claimed in claim 1, wherein the fillers have a mean particle size of 0.005-2.0, preferably 0.01-0.5, μm.

11. The dental material as claimed in claim 1, wherein as component III it contains nanoparticulate or microfine fillers, preferably pyrogenic silica, precipitated silica or X-ray-opaque fillers or mixtures of these substances.

12. The dental material as claimed in claim 1, wherein component III contains ytterbium fluoride, tantalum (V) oxide or barium sulfate or mixtures of these substances as filler.

13. The dental material as claimed in claim 11, wherein the content of the nanofiller having a mean particle size ≦40 μm in the monomer is 2-10% by weight, preferably 5-10% by weight, based on the monomer.

14. The dental material as claimed in claim 1, wherein in component III it contains organic fillers or contains composite fillers, which contain a monomer or monomer mixture and one or more inorganic fillers.

15. The dental material as claimed in claim 14, wherein the inorganic fillers contained are ground glass or SiO₂ modifications or X-ray-opaque fillers.

16. The dental material as claimed in claim 15, wherein the fillers have a particle size of 0.1-50 μm, preferably 0.2-20 μm.

17. The dental material as claimed in claim 1, wherein the filler or the filler mixture in accordance with component 111 has a refractive index of between 1.45 and 1.55, preferably between 1.46 and 1.52.

18. The use of the curable dental materials as claimed in claim 1 for the preparation of cured dental materials having an adjustable translucence and high opalescence, in particular of highly esthetic filling materials.
